(19) United States
(12) Patent Application Publication
(10) Pub. No.: US 2020/0371039 A1
(43) Pub. Date: Nov. 26, 2020

(54) DETECTION OF PRODUCTION FLUID ADDITIVES USING SPIKING

(71) Applicant: LUX ASSURE LIMITED, Edinburgh (GB)

(72) Inventors: Scott RANKIN, Edinburgh (GB); Andrew OSNOWSKI, Edinburgh (GB); Fiona CARSON, Edinburgh (GB)

(21) Appl. No.: 16/636,213

(22) PCT Filed: Aug. 3, 2018
(86) PCT No.: PCT/GB2018/052238

§ 371 Date: Feb. 3, 2020

(30) Foreign Application Priority Data
Aug. 4, 2017 (GB) 1712574.1

(51) Int. Cl. G01N 21/85; G01N 33/28; G01N 21/77; G01N 1/38; G01N 1/40

(52) U.S. Cl. CPC G01N 21/85; G01N 33/2823; G01N 21/77; G01N 2021/8411; G01N 1/4077; G01N 2021/8571; G01N 1/38

(57) ABSTRACT

A method of detecting production fluid additives in a fluid conducting and containment system when the additive is below its effective dose. The method includes adding an additional surfactant containing chemical to a fluid until the point of micelle formation in order to determine the amount of additive in a system when it is below its effective dose.

The invention relates to the detection of production fluid additives when in a system below their effective dose. More particularly, the invention relates to the detection of biocides or other micelle-forming production chemicals in production fluids when in a system below their effective dose.

In the oil and gas industry, various additives are added to production fluids. For example, corrosion inhibitors, biocides, foamers, defoamers, paraffin control agents, emulsifiers, demulsifiers, anti-swelling agents, hydrate inhibitors, anti-caking agents, scale dissolvers, wetting agents, and wax control agents. These additives are added at various levels depending on their function and expense.

Organic film-forming corrosion inhibitors are a widely used additive in oilfield systems and are also commonly used in oil and gas processing and petrochemical industries. The active ingredient is usually a detergent-like or surfactant molecule with a charged polar (i.e. water-soluble or hydrophilic) head group and an uncharged non-polar (i.e. oil-soluble or lipophilic) tail. When introduced into a pipe, these compounds partition to interfaces where the opposite electrostatic properties of each part of the molecule create least energetic repulsions. In practice, the molecules adsorb to the surface of the pipe, and form ordered structures on the surface thereby creating a protective film.

Surfaces requiring protection include pipes, conduits, tubes, and other metal fixtures and any component in regular contact with corrosive fluids. These components may be used in exploration, drilling, completion, production operations, refining, water treatment systems and/or transportation of produced fluids, products or intermediates.

Corrosion is a growing problem particularly for older oil wells, since the composition of produced fluids changes from predominantly hydrocarbons to hydrocarbon/brine mixtures to predominantly brine with lower hydrocarbon yields. The average age of producing wells is increasing and so the capacity for corrosion increases too. On average, it is estimated that three barrels of water are produced for every barrel of oil produced globally. Gas wells also suffer from increasing corrosion with age due to production of corrosive constituents. Deliberate transport of potentially corrosive fluids, such as dissolved carbon dioxide, for carbon sequestration, and extraction of petroleum sources such as acid crude and highly sour gas condensates, is also becoming more common and is likely to increase further in the future.

Corrosion inhibitor use is the first line of defence for many assets and it is important to ensure adequate corrosion inhibitor availability: to ensure chemical reserve is available, use back up pumps, check injection vs. production rates frequently, ensure there is nothing which might adversely affect the chemical, such as loss to solids.

Traditional corrosion inhibitor residual monitoring can help monitor this and flag concerns, but is fraught with difficulties.

The need for residual monitoring is because active corrosion inhibitors may be consumed in the inhibition process or lost due to deposit and corrosion products, as well as chemical degradation processes, partitioning and combinations of such phenomena. It is important that adequate availability is maintained.

Corrosion inhibitor formulations are complex chemical mixtures and difficult to monitor because of the large number of components involved. Few monitoring methods measure all components of a formulation. For example, a colourimetric approach may be used which is based on the detection of a colour produced from the reaction of compounds with amines. However, this approach is limited to the monitoring of specific classes of chemicals. Alternatively, in the ion pair technique, an excess of a large anionic molecule is added to the water containing a cationic corrosion inhibitor. The ion pair formed is then extracted into a solvent and its concentration determined colourimetrically. A disadvantage of the ion pair technique is that the method suffers from interferences from other species and is restricted to formulations with known chemical composition and so needs to be tailored to the components of the mixture.

Ultraviolet (UV) absorption methods that are based upon the measurement of the absorbance of UV light by a component of a corrosion inhibitor formulation may also be used. Fluorescence methods are also available, these methods use the fluorescence spectra or emission intensities of specific inhibitors. These methods are prone to error from other absorbent or fluorescent species.

Other techniques can provide more information about the concentration of particular components in a fluid sample, such as those involving inductively coupled plasma (ICP) or mass spectrometry. However, these methods are time-consuming, use expensive and bulky equipment that is not suitable for on-site manipulation, and requires regular maintenance and uses expensive consumables such as columns. They must be handled by skilled technicians and can only provide quantitative results if following strict procedures. Less sophisticated mass spectrometry variations can be used but they are less informative and are still complicated and laboratory based. In addition, mass spectrometry usually requires the chemical composition of the formulation to be determined during the analysis and the method must be modified and tailored to the components of the composition. However, chemical companies rarely release information on the exact components of their corrosion inhibitor formulation and service companies often bind operators to “non-analysis agreements” to specifically stop them from analysing their formulations for chemical composition. Interpretation of results can therefore be difficult.

Many of the techniques described above can only be used in aqueous systems and are unsuitable for fluids comprising any significant amount of oil. The specific abilities will be slightly different for each different technology. False increases or decreases in signal from interferences means levels have significant uncertainty. All the techniques are susceptible to interferences and the components of interest often need to be extracted to be analysed. The extraction process can be time consuming and technically difficult. In addition, inhibitor may be lost during the extraction process.

Applicant's own publication WO2010/007397 describes an improved methodology for the detection of production fluid additives, including corrosion inhibitors, that uses the relationship between an additive at its effective dose and the presence of micelles. Micelle detection methods do not require detailed information on the chemical composition of the corrosion inhibitor formulations and the need for an extraction process is minimised. Therefore, these methods can be performed on-site, by relatively unskilled personnel, unlike more complex analytical chemistry techniques e.g. liquid chromatography-mass spectrometry (LC-MS).

Only one species of a corrosion inhibitor formulation is detected by colourimetric complex methods, whereas micelle formation encompasses multiple species. This can be of great importance, where constituent species may partition or adsorb differently to other components and when multiple surfactants are present. Micelles are, therefore, a better gauge of corrosion inhibitor availability in the sample and a method that works on creating micelles can account for sample effects better than existing approaches.

Direct fluorescence methods, whereby a component of the corrosion inhibitor formulation is fluorescent, suffers from interference from intrinsically-fluorescent hydrocarbons. This is also an issue for many colourimetric methods, which react with an active species to produce a fluorescent product or sample contents interfere e.g. chloride. In both cases, removal of solids and oil from the sample may be required. As well as introducing additional steps, corrosion inhibitor can be lost in the process of removal e.g. to filters, rendering data inaccurate. Micelle measurement is more robust, the optical marker can be chosen to generate fluorescence away from that of the hydrocarbon. Background readings before addition of the marker can account for transmittance variation, due to the presence of solids, oil or emulsions and background fluorescence readings can also be taken and subtracted from the final reading, after addition of the optical marker.

Additionally, some residual methods wash solids with solvents and include this in residual reporting. This may be necessary if samples have been sent to a laboratory for analysis and more precipitates have formed in transit, which is common with oilfield fluids. However, those solids in the sample which were formed prior to transit may be coated with inhibitor that should not be included in analysis, as it is unavailable to protect the pipe. However, by washing the solids all signal will be included, leading to an over reporting of chemical. Micelle measurements done on-site would not wash solids.

Despite the above-mentioned need to maintain the amount of corrosion inhibitors at an effective dose to maximise steel protection and minimise over use of chemicals, it can often be the case that corrosion inhibitors are dosed at concentrations below the effective dose. The reasons for this are many fold but include:
economically driven decisions to use less additive i.e. because the higher corrosion level is acceptable in the modelling of the system e.g. when considering lifespan of an asset;
the effective dose of inhibitor causes operational difficulties, such as oil in water separation problems that cannot be allowed; and
some chemicals are batch-dosed and so injected at high dose, which then reduces over time causing the amount additive to fall below the effective dose before redosing occurs.

Even though there may be intentional underdosing of a system, an operator may still want to know how much corrosion inhibitor is in the system:
as a residual measurement to inform management;
to determine how much they might have to increase the dose to reach the critical micelle concentration (CMC), without changing dosage on-site (because of possible repercussions); or
because they are interested in knowing on a regular basis (daily, weekly, monthly, quarterly, annually) how the concentration of a corrosion inhibitor changes over time because, for example, the effects of changes to production, water cut, solids production, or efficiency of injection pumps.

Considering the above, there is a need for a method that provides information on the amount of corrosion inhibitor in the fluid. The method needs to be simple, rapid and applicable without the need for expensive equipment. The need for extractions must be minimised and the method should be performable on-site, for example, on an oil platform or other oil extraction or production site. Other than being micelle-forming, the method needs to be independent of the particular chemical formulation of the corrosion inhibitor so that it can be widely applicable.

In the context of this patent application a dose refers to a quantity of a production fluid additive added to a system at a given time and an effective dose is a dose of an additive added to a system at a given time that is at a concentration which elicits the optimal effect of an additive, e.g. a corrosion inhibitor added at a concentration that provides the optimal corrosion inhibition.

It is an object of the invention to seek to mitigate problems such as those described above. In a first aspect of the invention there is provided, a method of detecting production fluid additives in a fluid conducting and containment system comprising a) taking a sample of a production fluid comprising an additive from the system, b) adding an optical marker and detecting an optical signal from a marker solution in the presence of micelles, c) if no micelles are detected, adding an additional micelle forming surfactant-containing chemical to the sample before, at the same time as, or after the marker solution until a micelle-related signal is generated, and d) determining the amount of additive in the sample as a function of the additional micelle forming surfactant-containing chemical added to the sample. This method is advantageous as it allows a user to determine the amount of an additive in a production fluid from the amount of additional surfactant-containing chemical that has to be added to the fluid in order to form micelles, which are an indication that the additive is at its effective dose.

Preferably, the additive is a corrosion inhibitor. Corrosion inhibitors are critical to the proper functioning of a system and are also an expensive additive. It is therefore the case that corrosion inhibitors may be added to a system below their effective dose but at an acceptable dose. However, due to their importance to the system it will still be of interest to the operator of the system to determine their exact amount within the system to make sure that they are at an amount that can perform their function acceptably.

Preferably, the method further comprises the step of determining the critical micelle concentration for the additive being added to the system prior to step a). If it is not already known, then determining the critical micelle concentration for a production fluid additive allows the amount of the additive in the system to be calculated.

Preferably, the method further comprises the step of diluting the sample prior to step a). Dilution of the sample reduces interferences from other species.

Preferably, the additional micelle forming surfactant-containing chemical added to the sample is the additive in step a). This is advantageous as adding new chemicals to that currently in the system may have unexpected consequences, for example, they may have different partitioning effects, and this may lead to inaccurate results.

Preferably, the fluid conducting and containment system is a system used to screen, test, produce and process oil and gas, and their products. These systems require additives, such as corrosion inhibitors in order to continue to function at peak capacity and it is therefore crucial to monitor the amount of the additives in order to ensure proper function of the systems.

Preferably, micelles formation is monitored using laser diffraction, interferometry or imaging, spectroscopic means, hyperspectral imaging or flow cytometry. These detection methods provide a quick and efficient methodology of detecting micelles.

Preferably, sampling is performed at one or more locations in the system. Oil and gas pipelines may involve a complex network of pipelines and may also be several miles long in places. Therefore, it is advantageous to take samples from different locations within the system as it allows for determination of the amount of the additive at various points within the system and a more accurate picture of the system as a whole.

Preferably, the method of the first aspect of the invention further comprises the step of preparing at least one control sample. Control samples may be used to assess the fluid and ensure representative data.

Preferably, salt is added to at least one of the control samples in order to assess the ionic strength of the sample. This is advantageous as it ensures micelles can be created and there is not an issue with the fluid being of low ionic strength.

Preferably, an additional micelle-forming chemical is added to at least one of the control samples in order to assess if the sample contains a component that prevents the formation of micelles. This is advantageous as it ensures that there is nothing in the fluid that prevents micelle formation.

According to a second aspect of the invention, there is provided a kit for performing the method of the first aspect of the invention comprising at least one marker solution containing an optically detectable marker. In some embodiments the kit may further comprise positive and negative control, reference standards, a means to measure transmission of samples, a means to measure pH of samples, a means to mix sample and marker, a means to filter samples, and a means to centrifuge samples. In other embodiments, the kit may further comprise instructions for performing the method and/or a micelle forming surfactant-containing chemical.

The invention will be further described with reference to the drawings and figures, in which:

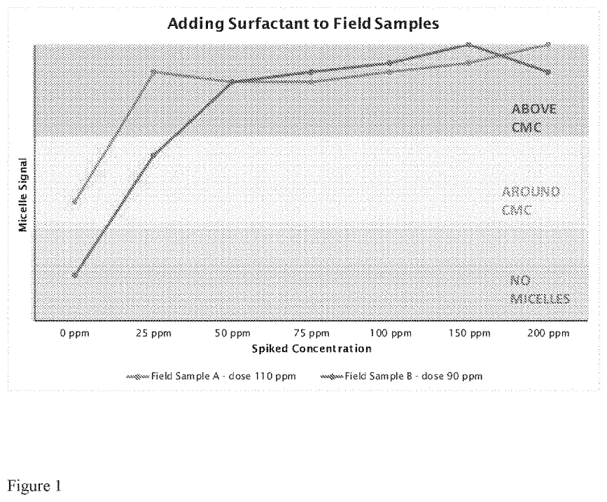

FIG. 1 shows a graph of optical signal intensity verses the amount of additive added to two field samples between 0-200 ppm; and

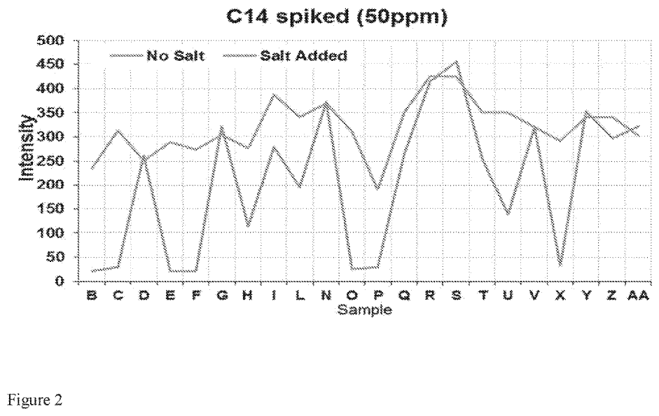

FIG. 2 shows a graph of optical signal intensity after adding micelle forming surfactant to samples containing corrosion inhibitor to enable micelle detection at the critical micelle concentration.

### EXPERIMENT A: ADDING SURFACTANT CHEMICAL TO FIELD SAMPLES TO ENABLE MICELLE DETECTION

The following experiments were conducted on-site at an oil and gas processing facility. Spiking in ≥25 ppm of surfactant chemical, used to mitigate corrosion, into Field Sample A (110 ppm dose rate) resulted in a strong micelle signal, indicating the chemical was present in the spiked sample at a concentration above the CMC (FIG. 1). When the dose was lowered to 90 ppm no micelles were detected in Field Sample B. Spiking 25 ppm into Field Sample B, showed a signal around the CMC, while spiking in ≥50 ppm resulted in a chemical concentration in the spiked sample B above the CMC (FIG. 1). A dose of around 110-135 ppm would be expected to result in the presence of chemical above the CMC for this area of the system. As the dosage of chemical into the system was reduced the amount of chemical required to be added to the sample to detect micelles was increased.

Experiment B. Adding chemical to samples to demonstrate the impact fluid changes can have on the CMC

˜50 mL of brine was added to a 100 mL volumetric flask which was then placed on a balance and weighed. 100 μL of a commercially available formulated corrosion inhibitor (Aliphatic amine derivative) was added to the flask and the flask re-weighed to determine the mass of inhibitor added. The contents were gently swirled to ensure mixing without foam generation, the volume was adjusted to the graduated mark with the relevant brine, the flask capped and inverted carefully (˜10 times) to ensure homogeneity of the prepared 1,000 ppm solution. Each of the inhibitor-salt solutions were homogenous, with no droplet precipitation or haze observed at the time of use. The 1,000 ppm inhibitor solution was diluted with further diluent brine to give final concentrations of inhibitor. To 1980 μL of the inhibitor brine solution was added 20 μL of optical marker (Nile Red) through a positive displacement pipette. The cuvette was capped securely and inverted gently (˜5 times) to ensure homogeneity of solution but without causing the mixture to foam. Fluorescence readings were taken immediately upon complete mixing. The concentration of brine had a significant impact on critical micelle concentration:

[symbol] 40,000 ppm chloride ion (CaCl₂). CMC 41 ppm

[symbol] 20,000 ppm chloride ion (CaCl₂), CMC 51 ppm

In field fluids, the amount of micelle-forming surfactant that may be required to be added to a sample to form micelles will vary. This will reflect changes in the field fluids and micelles can help capture information on the influence of such changes on corrosion inhibitor effectiveness or availability.

22 produced fluids were centrifuged prior to use (6000 rpm for 1 hour). After centrifugation, 10 mL of supernatant from each fluid was transferred to a glass vial and 50 μL of 10,000 ppm Myristyltrimethylammonium bromide (micelle forming surfactant; MTAB) was added. This gave a 50 ppm MTAB solution for each of the produced fluids. The spiked samples were mixed well by inversion and then allowed to settle for ca 1 hour. After 1 hour, 5 mL of each spiked fluid was transferred to separate glass vials—each containing 0.29 g of NaCl. This amount of NaCl gave a 1 M NaCl brine, assuming no other salts were present in the produced fluid. The samples were mixed gently until all the salt dissolved and then left to settle for ca 1 hour. The 50 ppm spikes, with and without salt, were subsampled for analysis by mixing 2 mL of sample with 20 μL of optical marker (Nile Red) and fluorescence determined, see FIG. 2.

For many samples micelle-related signal was very low, until the salt concentration was increased—B, E, F, O, P, X. Oil and gas field fluids can vary significantly: this might be in terms of water cut, salinity, production chemicals, type of hydrocarbon or solids. These differences can impact the critical micelle concentration which will be reflected in the amount of chemical required to be added to a sample before micelles are observed.

Experiment C: Adding micelle forming surfactant to samples containing corrosion inhibitor to enable micelle detection at the critical micelle concentration

The critical micelle concentration of a commercially available formulated corrosion inhibitor, whose primary ingredients were tall oil fatty acids and thioglycolic acid, was determined to be ˜25-30 ppm in 1M NaCl.

Solutions of 0, 5, 10, 15 and 20 ppm were prepared and then spiked with various amounts of the corrosion inhibitor until the CMC was reached. This was recorded compared to the CMC as originally determined. As shown in Table 1, all samples showed a good match i.e. the amount of chemical added plus the original concentration in the sample equalled the CMC determined for the chemical at the outset.

TABLE 1

| | Spike | | Calcu- | |
|---|---|---|---|---|
| Base | conc. | Total | lated | Differ- |
| conc. | for micelle | conc. | CMC | ence |
| (ppm) | (ppm) | (ppm) | (ppm) | (ppm) |
| 0 | 22 | 22 | 25 to 30 | −3 |
| 5 | 21 | 26 | 25 to 30 | In range |
| 10 | 15 | 25 | 25 to 30 | In range |
| 15 | 15 | 30 | 25 to 30 | In range |
| 20 | 4 | 24 | 25 to 30 | −1 |

The invention relates to the issue of how to determine the amount of a production fluid additive if it is in a system below the effective dose. The effective, or optimal, dose is an amount of additive at which the critical micelle concentration is reached. For example, if it is known or has been determined that the critical micelle concentration for an additive is 100 ppm, then knowing that 10 ppm needs to be added to a sample to form micelles means that the sample is at 90 ppm. The spiked chemical may be added to the aqueous or hydrocarbon phase of a sample. This is to best mimic partitioning and behaviour in the systems.

The approach described here may also be applied to understand the impact system changes have on chemical availability. For example, an operator believes solids are going to be produced and questions if the chemical additive will adsorb to them. A sample from the system is taken and it is determined if micelles are present, and if not, how much additive is needed to be added to form them. Solids are added to another sample and additive added in order to determine if more chemical, which would indicate loss of chemical to the solids surface, is required. The same could be done for other potential system changes, such as water cut, hydrocarbon type, production chemicals and brine strength.

Additive micelles may be detected in a number of ways. For example, an imaging approach may be used. Micelles are, by definition, not truly water-soluble and exist as dispersed liquid particles. It is therefore possible to observe corrosion inhibitor micelles by optical means. If large enough (i.e. greater than the Abbe limit of about 0.5 μm) then conventional microscopic imaging is possible and the images can be analysed using particle analysis software. Other optical means may also be used depending on the properties of the micelles.

In one embodiment, a compound capable of associating with a micelle to produce an amplified or detectable signal may be added. For example, a marker solution may be added to the fluid which creates or enhances a detectable property (e.g. fluorescence). The signal is amplified when associated with the micelle relative to the disassociated state and therefore increases the signal to noise ratio resulting in increased overall sensitivity. The alteration in signal might, for example, result from a change in the electronic environment of the marker molecule which varies the molecular dipole moment in the ground and excited states. These differences result in a relative modification of the quantised energy of light absorbed or emitted in spectroscopic processes and so can be measured experimentally, for example through absorption, transmission, fluorescence intensity, fluorescence wavelength, fluorescence polarisation or fluorescence lifetime. Examples of suitable optical markers are meropolymethines, pyridinium-N-phenolate betaines, phenoxazones, N,N-dialkylaminonaphthalenes, N,N-dialkylaminostyrenes, N,N-dialkylaminonitrobenzenes, coumarins, N,N-dialkylindoaniline, vinylquinoliums, arylaminonaphthalene sulfonates and 9-diethylamino-5-benzo[α]phenoxazinone (Nile Red).

Due to these changes being strongly influenced by the polarity of the surrounding matrix, measurement of the light can be a probe for chemical environment. Alternatively, the marker may only be soluble in the micelle and solubility may determine whether a signal is generated or not, in either such case the signal may be colourimetric, absorbance, luminescent or fluorescent. Generally, UV and fluorescence measurements are faster than colourimetric alternatives which require an extraction step.

Micelles have distinct optical properties of shape and light diffusion, diffraction and reflection which allow them to be discriminated from other particles. Smaller particles may be imaged beyond the diffraction limit using, for example, dark-field imaging and/or Brownian motion analysis.

Another method that may be used for detecting and analysing the micelles is spectral analysis (spectroscopy). In complex fluids, such as those from oilfield production, there are likely to be a number of components arising from non-corrosion inhibitor origins which must be discriminated against in the analysis. One method of achieving this is by interrogating the analyte with light and recording the resulting spectral properties of the system. In one embodiment this may involve recording the bulk UV, visible or infrared absorption of light at a certain wavelength. The resulting absorption, either with or without the addition of a marker solution, may be indicative of the presence of micelles.

Alternatively, fluorescence emission, lifetime or polarisation could be used.

In an expansion on this, spectral resolution can be combined with an imaging system so that each recorded pixel will contain spectral information rather than just intensity. For example, fluorescence imaging can be used to measure the colour of the fluorescence emission, the colour emitted in response to the presence of corrosion inhibitor being different from the colour emitted in response to the presence of, for example, oil, sand or other additives. These methods can be broadly termed as spectral or hyperspectral imaging. In one embodiment, the spectrum imaged may just be a simple recording at three different wavelengths e.g. RGB, or it could include a full spectral scan across e.g. 500-900 nm.

Diffraction technologies may also be used to detect and monitor the micelles. Systems for measuring nano-particles involving light scattering or diffraction techniques may be used to determine the particle size of the micelles in solution and also the properties of those particles. In its simplest form the diffraction of light resulting from suspended particles in solution can be used to determine the presence, average particle size and the relative distribution of particles in the solution. Addition of supplementary sensing technology such as interferometry, impedance and zeta potential measurements can additionally characterise the system to provide discrimination between micelles and interfering oilfield species.

Other methods for detecting and monitoring micelle formation are based on particle interrogation and counting systems. For example, flow cytometry is a method of examining and sorting microscopic particles in a fluid. These systems are built to varying specifications and record parameters including particle volume, shape, size etc. They are often also associated with fluorescence detection in microbiological studies and combine this with light scatter analysis in systems such as a Fluorescence-Activated Cell Sorter (FACS). Such a device could be modified to measure micelles in material to provide a rich pool of data. Because micelle detection requires no antibody binding step the analysis would also be much faster than traditional flow cytometry and may be amenable to offshore use.

Useful information may be obtained from monitoring micelle formation. Indeed, the amount of micelles in the fluid is related to the degree of corrosion inhibition and efficiency of the inhibitor. In addition, analysis of the micelles (e.g. assessment of their number, size and shape) will provide information on the physico-chemical properties of the fluid. As stated above, there is a link between the critical micelle concentration and the optimal corrosion inhibition.

1. A method of detecting production fluid additives in a fluid conducting and containment system comprising:
a) taking a sample of a production fluid comprising an additive from the system;
b) adding an optical marker and detecting an optical signal from a marker solution in the presence of micelles;
c) if no micelles are detected, adding an additional micelle forming surfactant-containing chemical to the sample before, at the same time as, or after the marker solution until a micelle-related signal is generated; and
d) determining the amount of additive in the sample as a function of the additional micelle forming surfactant-containing chemical added to the sample.

2. The method according to claim 1, wherein the additive is a corrosion inhibitor.

3. The method according to claim 1, further comprising the step of determining the critical micelle concentration for the additive being added to the system prior to step a).

4. The method according to claim 1, further comprising the step of diluting the sample prior to step a).

5. The method according to claim 1, wherein the additional micelle forming surfactant-containing chemical added to the sample is the additive in step a).

6. The method according claim 1, wherein the fluid conducting and containment system is a system used to screen, test, produce and process oil and gas, and their products.

7. The method according claim 1, wherein micelle formation is measured using laser diffraction, interferometry or imaging, spectroscopic means, hyperspectral imaging or flow cytometry.

8. The method according claim 1, wherein the fluid comprises one or more of water, oil, solids, gas, liquefied gas and/or emulsions.

9. The method according claim 1, wherein sampling is performed at one or more locations in the system.

10. The method according claim 1, further comprising the step of preparing at least one control sample.

11. The method according to claim 10, wherein salt is added to at least one of the control samples in order to assess the ionic strength of the sample.

12. The method according to claim 10, wherein an additional micelle-forming chemical is added to at least one of the control samples in order to assess if the sample contains a component that prevents the formation of micelles.

13. A kit for performing the method of claim 1 comprising:
at least one marker solution containing an optically detectable marker.

14. The kit according to claim 13, further comprising:
positive and negative controls.

15. The kit according to claim 13, further comprising:
reference standards.

16. The kit according to claim 13, further comprising:
a means to measure transmission of samples.

17. The kit according to claim 13, further comprising:
a means to measure pH of samples.

18. The kit according to claim 13, further comprising:
a means to filter samples.

19. The kit according to claim 13, further comprising:
a means to centrifuge samples.

20. The kit according to claim 13, further comprising:
instructions for performing the method.

21. The kit according to claim 13, further comprising:
a micelle forming surfactant-containing chemical.
